(19) United States
(12) Patent Application Publication
(10) Pub. No.: US 2026/0294302 A1
(43) Pub. Date: Oct. 1, 2026

(54) METHOD AND SYSTEM FOR STRESS DETECTION BASED ON CARDIOVASCULAR PHYSIOLOGICAL SIGNALS

(71) Applicant: National Tsing Hua University, Hsinchu (TW)

(72) Inventors: CHI-CHUN LEE, Hsinchu (TW); Pin-Jhao Chen, Hsinchu (TW); Woan-Shiuan Chien, Hsichu (TW)

(21) Appl. No.: 19/313,739

(22) Filed: Aug. 28, 2025

(30) Foreign Application Priority Data
Mar. 31, 2025 (TW) 114112340

(51) Int. Cl. A61B 5/16; A61B 5/00; A61B 5/024

(52) U.S. Cl. CPC A61B 5/165; A61B 5/02405; A61B 5/7228; A61B 5/7264; A61B 5/7275

(57) ABSTRACT

A method for detecting stress based on cardiovascular physiological signals, and a system using the method are provided. The method includes: obtaining multiple signal segments based on a cardiovascular physiological signal, where the signal segments are stationary under an augmented Dickey-Fuller (ADF) test; calculating multiple trends for the signal segments; obtaining a detrended signal corresponding to the cardiovascular physiological signal based on the trends; extracting multiple first heart rate variability (HRV) features based on the detrended signal; and obtaining a stress detection result corresponding to the cardiovascular physiological signal based on the first HRV features.

### CROSS-REFERENCE TO RELATED APPLICATION(S)

The present disclosure claims the benefit of and priority to Taiwan Patent Application No. 114,112,340, filed on Mar. 31, 2025, the content of which is hereby incorporated herein fully by reference into the present disclosure for all purposes.

### FIELD

The present disclosure generally relates to a technology for processing and analyzing biological signals, and more particularly, relates to a method and system for detecting stress, based on cardiovascular physiological signals, by combining signal processing and machine learning.

### BACKGROUND

In modern healthcare systems, physiological biosignals, such as electrocardiography (ECG) and electroencephalography (EEG) are widely used for automatic assessment and prediction. However, these studies often face the challenge of individual identity bias. When a dataset includes multiple samples from the same subject, the signals often carry individual physiological characteristics, making it difficult for the model to distinguish between personal traits and task-related features. For example, heart rate variability in ECG or spectral features in EEG may remain consistent across different contexts, leading the model to misinterpret individual characteristics as task-relevant signals, thereby reducing the model's generalization ability.

Decoupling techniques have been widely applied to separate components of interest from various signals. Methods based on autoencoders (AE) and variational autoencoders (VAE) are widely used in the field of speech recognition to extract speaker features and improve system effectiveness. Similarly, in music signal processing, VAEs are used for feature extraction and style transfer. However, these techniques heavily rely on large datasets, and their generalization performance degrades significantly when the number of samples is limited. The limitation is particularly critical in the context of physiological biosignals, where sample sizes are typically small.

Currently, no method is capable of effectively separating individual features from task-related features, especially in biomedical domains with limited sample sizes. Therefore, developing an effective decoupling method under conditions of limited data is crucial to improving the accuracy and generalizability of physiological biosignal analysis.

### SUMMARY

The present disclosure is directed to a method and system for detecting stress based on cardiovascular physiological signals, which are capable of effectively decoupling the cardiovascular physiological signals, thereby improving the accuracy of stress detection.

According to a first aspect of the present disclosure, a method for detecting stress based on cardiovascular physiological signals is provided. The method includes: obtaining multiple signal segments based on a cardiovascular physiological signal, the signal segments being stationary, as determined by an augmented Dickey-Fuller (ADF) test; calculating multiple trends for the signal segments; obtaining a detrended signal corresponding to the cardiovascular physiological signal based on the trends; extracting multiple first heart rate variability (HRV) features based on the detrended signal; and obtaining a stress detection result corresponding to the cardiovascular physiological signal based on the first HRV features.

In an implementation of the first aspect, the method further includes: obtaining multiple candidate modulated signals, based on the cardiovascular physiological signal, using multiple modulation schemes; obtaining multiple second HRV features extracted from a modulated signal selected from the candidate modulated signals; and obtaining the stress detection result corresponding to the cardiovascular physiological signal based on the first HRV features and the second HRV features.

In another implementation of the first aspect, obtaining the second HRV features extracted from the modulated signal selected from the candidate modulated signals includes: extracting multiple second candidate HRV features respectively from the candidate modulated signals; obtaining multiple weights corresponding to the second candidate HRV features using a selection model; and selecting one of the second candidate HRV features that corresponds to a maximum weight of the multiple weights. The selected one of the second candidate HRV features is extracted from the modulated signal.

In another implementation of the first aspect, each modulation scheme includes at least one of magnitude warping, scaling, noise addition, jittering, time warping, window warping, and permutation.

In another implementation of the first aspect, obtaining the stress detection result corresponding to the cardiovascular physiological signal based on the first HRV features and the second HRV features includes: obtaining embeddings using a multi-head attention model based on the first HRV features and the second HRV features; and obtaining the stress detection result based on the embeddings.

In another implementation of the first aspect, the multi-head attention model is trained using a guided contrastive learning method.

In another implementation of the first aspect, obtaining the signal segments based on the cardiovascular physiological signal includes: normalizing the cardiovascular physiological signal to obtain a normalized signal; segmenting the normalized signal into multiple first candidate segments based on a first segment length; determining whether all of multiple first p-values, each corresponding to one of the first candidate segments, are smaller than a predefined threshold using the ADF test; determining that the first candidate segments are the signal segments when all of the first p-values are smaller than the predefined threshold; and re-segmenting the normalized signal into multiple second candidate segments using a second segment length when at least one of the first p-values is not smaller than the predefined threshold.

In another implementation of the first aspect, the first segment length is shorter than the second segment length.

In another implementation of the second aspect, calculating the trends for the signal segments includes: fitting the trends, based on the signal segments, using an ordinary least squares (OLS) method.

According to a second aspect of the present disclosure, a system for detecting stress based on cardiovascular physiological signals is provided. The system includes at least one processor and at least one non-transitory computer-readable medium coupled to the at least one processor. The at least one non-transitory computer-readable medium stores one or more computer-executable instructions that, when executed by the at least one processor, cause the system to perform the method described in the first aspect.

### BRIEF DESCRIPTION OF THE DRAWINGS

Aspects of the example disclosure are best understood from the following detailed description when read with the accompanying figures. Various features are not drawn to scale. Dimensions of various features may be arbitrarily increased or reduced for clarity of discussion.

FIG. 1 is a diagram illustrating a system for detecting stress, according to an example implementation of the present disclosure.

FIG. 2 is a flowchart illustrating a method for detecting stress, according to an example implementation of the present disclosure.

FIG. 3 is a diagram illustrating signal detrending, based on an augmented Dickey-Fuller (ADF) test, according to an example implementation of the present disclosure.

FIG. 4 is a flowchart illustrating a method for detecting stress, according to an example implementation of the present disclosure.

FIG. 5 is a diagram illustrating a method for detecting stress, according to an example implementation of the present disclosure.

FIG. 6 is a block diagram illustrating a calculating system, according to an example implementation of the present disclosure.

### DETAILED DESCRIPTION

The following description contains specific information pertaining to exemplary implementations in the present disclosure. The drawings in the present disclosure and their accompanying detailed description are directed to merely exemplary implementations. However, the present disclosure is not limited to merely these exemplary implementations. Other variations and implementations of the present disclosure will occur to those skilled in the art. Unless noted otherwise, like or corresponding elements among the figures may be indicated by like or corresponding reference numerals. Moreover, the drawings and illustrations in the present disclosure are generally not to scale, and are not intended to correspond to actual relative dimensions.

For the purpose of consistency and ease of understanding, like features are identified (although, in some examples, not shown) by numerals in the example figures. However, the features in different implementations may be differed in other respects, and thus shall not be narrowly confined to what is shown in the figures.

References to “one implementation,” “an implementation,” “example implementation,” “various implementations,” “some implementations,” “implementations of the present application,” etc., may indicate that the implementation(s) of the present application so described may include a particular feature, structure, or characteristic, but not every possible implementation of the present application necessarily includes the particular feature, structure, or characteristic. Further, repeated use of the phrase “in one implementation,” or “in an example implementation,” “an implementation,” do not necessarily refer to the same implementation, although they may. Moreover, any use of phrases like “implementations” in connection with “the present application” are never meant to characterize that all implementations of the present application must include the particular feature, structure, or characteristic, and should instead be understood to mean “at least some implementations of the present application” includes the stated particular feature, structure, or characteristic. The term “coupled” is defined as connected, whether directly or indirectly through intervening components, and is not necessarily limited to physical connections. The term “comprising,” when utilized, means “including, but not necessarily limited to”; it specifically indicates open-ended inclusion or membership in the so-described combination, group, series and the equivalent.

Additionally, for the purposes of explanation and non-limitation, specific details, such as functional entities, techniques, protocols, standard, and the like are set forth for providing an understanding of the described technology. In other examples, detailed description of well-known methods, technologies, system, architectures, and the like are omitted so as not to obscure the description with unnecessary details.

The terms “first,” “second,” and “third,” etc. used in the specification and the accompanying drawings of the present disclosure are intended to distinguish between different objects, rather than to describe a particular order. In addition, the term “comprising” and any variations thereof are intended to cover non-exclusive inclusion. For example, a process, method, system, product, or apparatus that includes a series of operations/steps or modules is not limited to the listed operations/steps or modules but may optionally include additional operations/steps or modules not listed, or optionally include additional operations/steps or modules inherent in such processes, methods, products, or apparatus.

The following description is provided in conjunction with the accompanying drawings to illustrate implementations of the present disclosure.

The present disclosure provides a system and method for detecting stress, which may decouple cardiovascular physiological signals to reduce interference between identity information and task-related information within the cardiovascular physiological signals, thereby improving the accuracy of stress detection. It should be noted that in several implementations of the disclosure, a heart rate (HR) signal is used as an example of the cardiovascular physiological signal to illustratively explain the technology and effects of the disclosure. However, the disclosure is not limited thereto. Those skilled in the art may apply the methods proposed in the disclosure to other types of cardiovascular physiological signals based on the technical concepts introduced in these implementations.

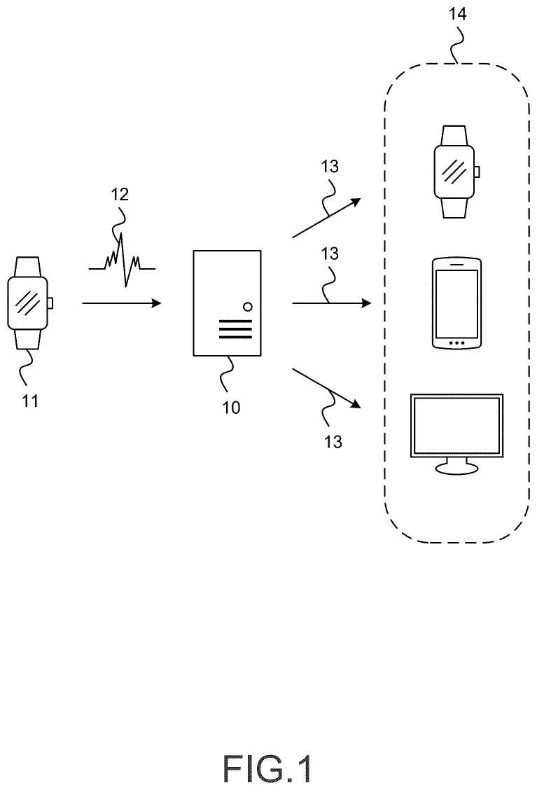

FIG. 1 is a diagram illustrating a system for detecting stress, according to an example implementation of the present disclosure.

Referring to FIG. 1, the system 10 for detecting stress may obtain a cardiovascular physiological signal 12 from a first device 11. Based on the cardiovascular physiological signal 12, the system 10 may perform a method for detecting stress according to implementations of the present disclosure to obtain a stress detection result 13. In addition, the system 10 may send the stress detection result 13 to a second device 14.

In some implementations, the first device 11 may be an internal or external device of the system 10 for detecting stress. For example, the first device 11 may be a device including physiological signal measurement components such as an electrocardiograph (ECG), photoplethysmograph (PPG), sphygmomanometer, phonocardiograph (PCG), or cardiac output monitor. For example, the first device 11 may include a wearable device, a portable device, and/or a fixed device.

In some implementations, the cardiovascular physiological signal 12 may include, but is not limited to, a heart rate signal.

In some implementations, the stress detection result 13 may include a binary result indicating presence or absence of stress. In some implementations, the stress detection result 13 may include a stress level and/or a stress score.

In some implementations, the second device 14 may be an internal or external device of the system 10 for detecting stress, including output components utilizing modalities, such as images or sounds. For example, the second device 14 may be a wearable device, a portable device, and/or a fixed device.

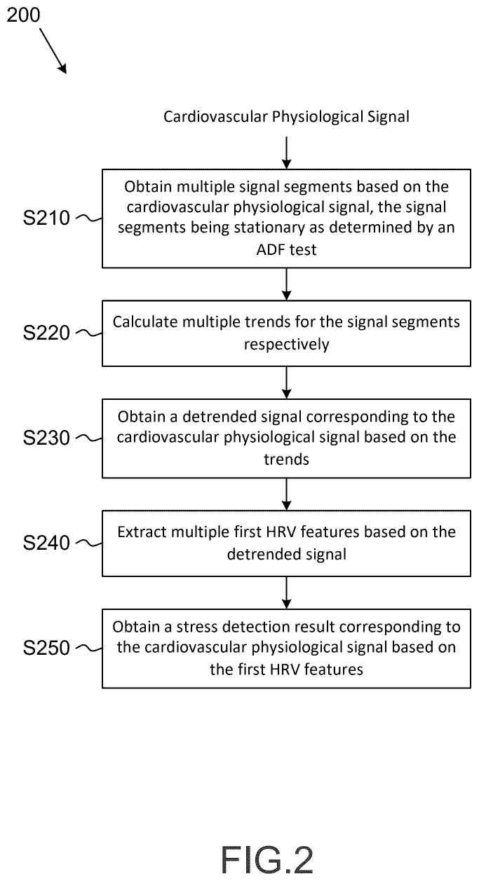

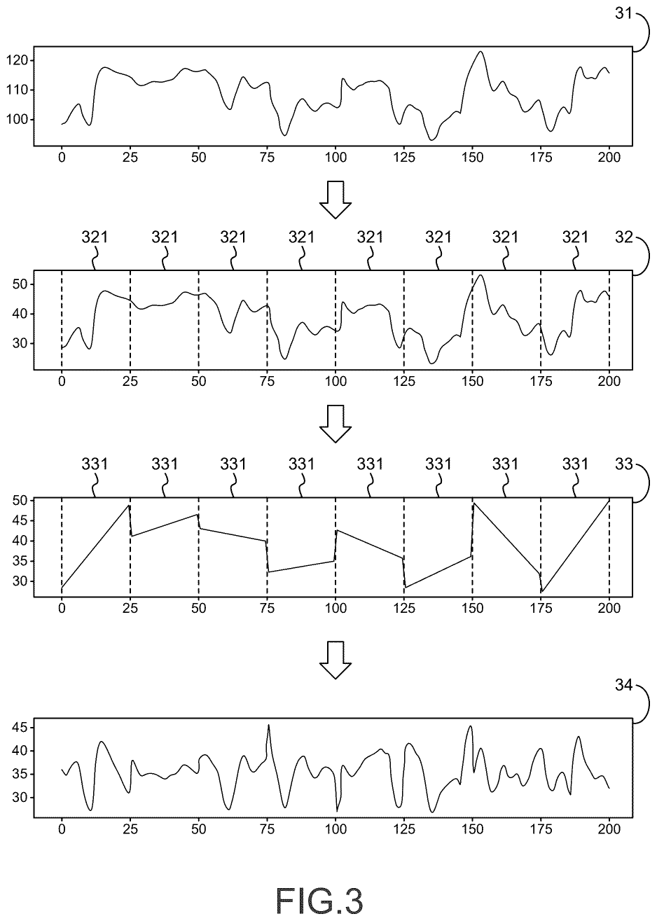

FIG. 2 is a flowchart illustrating a method for detecting stress, according to an example implementation of the present disclosure. FIG. 3 is a diagram illustrating signal detrending based on an augmented Dickey-Fuller (ADF) test, according to an example implementation of the present disclosure. The method for detecting stress in FIG. 2 may be illustrated by a process 200. The method may be performed by the system 10 for detecting stress of FIG. 1, and the process may include detrending of the cardiovascular physiological signal, based on the ADF test, as shown in FIG. 3. Therefore, the implementations related to FIG. 2 are described with FIGS. 1 and 3.

Referring to FIG. 2, the system 10 for detecting stress may first obtain the cardiovascular physiological signal 12. For example, the system 10 may obtain a heart rate signal from a wearable device 11.

In operation S210, the system 10 for detecting stress may obtain multiple signal segments based on the cardiovascular physiological signal 12, and the signal segments are determined stationary by an ADF test.

Specifically, the system 10 for detecting stress may segment the cardiovascular physiological signal 12 (e.g., the heart rate signal or a normalized signal thereof) into multiple signal segments with each being stationary, as determined by the ADF test.

In some implementations, referring to FIG. 3, the cardiovascular physiological signal 12 may be a heart rate signal 31. The system 10 for detecting stress may normalize the heart rate signal 31 to generate a normalized signal 32, and segment the normalized signal 32 into multiple signal segments 321. During the segmenting process, the system 10 for detecting stress may target that each signal segment 321 is stationary, as determined by an ADF test. When a p-value of a signal under the ADF test is smaller than a predefined threshold, a null hypothesis is rejected, meaning that a corresponding signal may be considered stationary. Otherwise, the null hypothesis is accepted, meaning that the signal may be considered non-stationary.

In some implementations, the signal segments 321 may each have the same length. In other implementations, the signal segments 321 may have different lengths.

In a case where each signal segment 321 has the same length, the system 10 for detecting stress may first segment the normalized signal 32 into multiple first candidate segments using a first segment length. For each of the first candidate segments, a p-value may be calculated using the ADF test. When all the p-values corresponding to the first candidate segments are smaller than the predefined threshold, the system 10 for detecting stress may determine the first candidate segments as the signal segments 321. When at least one of the p-values is not smaller than the predefined threshold, the system 10 for detecting stress may select a second segment length and segment the normalized signal 32 into multiple second candidate segments. When all the p-values corresponding to the second candidate segments are smaller than the predefined threshold, the second candidate segments may be determined as the signal segments 321. Otherwise, the system 10 for detecting stress may select a third segment length and repeat the above operations/steps until a set of signal segments 321 is found.

In some implementations, the segment lengths may be selected from small to large, starting from a predefined minimum length up to a predefined maximum length. In some implementations, the segment lengths may be selected from large to small, starting from a predefined maximum length down to a predefined minimum length.

In operation S220, the system 10 for detecting stress may calculate a trend for each of the signal segments.

Referring to FIG. 3, since each signal segment 321 is stationary, there may be a corresponding trend 331. In some implementations, the system 10 for detecting stress may apply an ordinary least squares (OLS) method to fit a trend 331 for each signal segment 321, thereby obtaining a trend line 33.

In operation S230, the system 10 for detecting stress may obtain a detrended signal corresponding to the cardiovascular physiological signal 12 based on the trends.

Referring to FIG. 3, the system 10 for detecting stress may subtract the trend lines 33 from the normalized signal 32 to obtain a detrended signal 34.

In some implementations, the detrended signal obtained by applying the ADF test-based detrending operation to the cardiovascular physiological signal (e.g., the heart rate signal 31) may be regarded as a signal that contains identity features within the cardiovascular physiological signal. In other words, by performing operations S210, S220, and S230, the system 10 for detecting stress may successfully extract individual- or identity-related features from the cardiovascular physiological signal, such as the detrended signal 34.

In some implementations, operations S210, S220, and S230 may be performed based on the control flow presented in the following pseudocode:

Require: signal X of length N
Ensure: detrended signal X_{detrended}
1: Step 1: Initial Setup
2: Initialize minimum allowed segment length Lₘᵢₙ and maximum allowed segment length Lₘₐₓ
3: Set tolerance for stationarity ∈
4: Step 2: Segment Detection
5: for L = Lₘᵢₙ to Lₘₐₓ do
6: Divide X into segments of length L
7: for each segment xᵢ do
8: Perform Augmented Dickey-Fuller (ADF) test on xᵢ
9: if p-value < ∈ then
10: Mark segment xᵢ as stationary
11: end if
12: end for
13: if all segments are stationary then
14: Proceed to Step 3
15: end if
16: end for
17: Step 3: Detrending
18: For each segment, calculate a local trend and remove it from X
19: Combine detrended segments to obtain X_{detrended}
20: return X_{detrended}

In operation S240, the system 10 for detecting stress may extract multiple first heart rate variability (HRV) features based on the detrended signal.

Specifically, the system 10 for detecting stress may use a feature extractor to extract multiple first HRV features from the detrended signal.

Advantageously, these first HRV features may be considered features that better reflect an individual's identity. In other words, for each heart rate signal, a set of first HRV features may be obtained. Multiple sets of first HRV features derived from different heart rate signals of the same individual may have high similarity. Thus, in the present disclosure, the first HRV features are also referred to as identity features.

In operation S250, the system 10 for detecting stress may obtain a stress detection result 13 corresponding to the cardiovascular physiological signal 12 based on the first HRV features.

Specifically, obtaining the first HRV features that reflect identity may provide additional individual-related information corresponding to the cardiovascular physiological signal. With the aid of this additional information, the system 10 may perform more accurate analysis for the target task. In other words, the system 10 for detecting stress may obtain a more accurate stress detection result 13.

In some implementations, the system 10 for detecting stress may first determine/identify the identity from the first HRV features using a pre-trained model, and retrieve a stress detection model associated with the identified identity (e.g., stored in advance). Afterwards, the system 10 may input the cardiovascular physiological signal 12 into the stress detection model associated with the identified identity to obtain the stress detection result 13. In other words, the system 10 for detecting stress may maintain different stress detection models for different identities, thereby reducing the impact of individual differences on stress detection.

In some implementations, the identity may be maintained in the system 10 for detecting stress in the form of an identifier or other representation to protect user privacy.

In some implementations, the system 10 for detecting stress may further use the first HRV features reflecting the identity to extract task-related features (e.g., second HRV features, also referred to as content features) from the cardiovascular physiological signal 12. Example implementations are described in detail below.

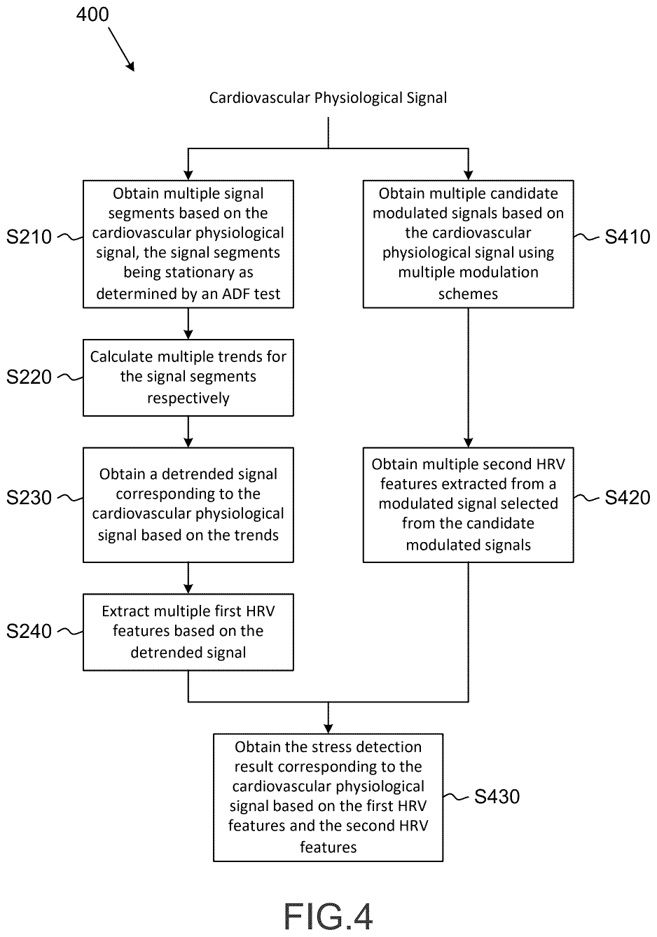

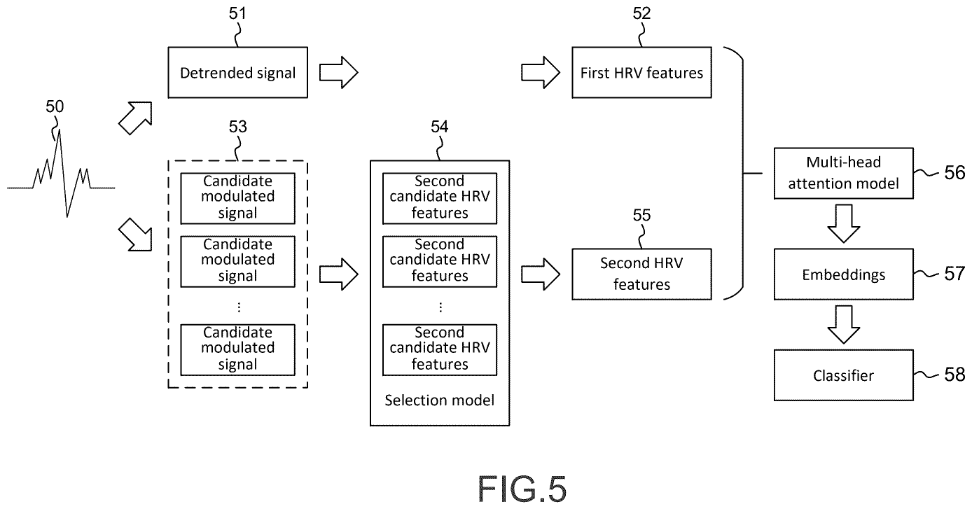

FIG. 4 is a flowchart illustrating a method for detecting stress, according to an example implementation of the present disclosure. FIG. 5 is a diagram illustrating a method for detecting stress, according to an example implementation of the present disclosure. The method for detecting stress in FIG. 4 may be illustrated by a process 400, where multiple operations correspond to those of process 200 and will not be redundantly described. Moreover, the method in FIG. 4 may be implemented by the system 10 for detecting stress, as shown in FIG. 1, and the models used in the method may be illustrated by the architecture, as shown in FIG. 5. Therefore, the implementations related to FIG. 4 will be described with FIGS. 1 and 5.

Referring to FIGS. 4 and 5, in operations S210 to S240, the system 10 for detecting stress may first obtain a detrended signal 51 corresponding to a cardiovascular physiological signal 50 and then extract multiple first HRV features (e.g., identity features) from the detrended signal 51.

In operation S410, the system 10 for detecting stress may obtain multiple candidate modulated signals, based on the cardiovascular physiological signal 12, using multiple modulation schemes.

Specifically, in real-life scenarios, heart rate signals may be influenced by various factors such as visceral pathology, user activity, and environmental temperature, leading to variations in timing and amplitude. To address such variations, the system 10 for detecting stress may apply multiple modulation schemes to the cardiovascular physiological signal 12 to generate multiple candidate modulated signals. Each modulation scheme may belong to one of the following categories: spatial modulation, temporal modulation, or spatiotemporal modulation. Each modulation scheme applied to the cardiovascular physiological signal 12 may result in one candidate modulated signal. For example, as shown in FIG. 5, the system 10 for detecting stress may obtain multiple candidate modulated signals 53 based on the cardiovascular physiological signal 50 (e.g., heart rate signal) using multiple modulation schemes.

In some implementations, spatial modulation scheme(s) may include at least one of magnitude warping, scaling, noise addition, and jittering.

In some implementations, temporal modulation scheme(s) may include at least one of time warping, window warping, and permutation.

In some implementations, spatiotemporal modulation scheme(s) may include at least one spatial modulation scheme and at least one temporal modulation scheme. For example, a spatiotemporal modulation scheme may be a composite modulation scheme that starts with a temporal modulation scheme followed by a spatial modulation scheme.

In operation S420, the system 10 for detecting stress may extract multiple second HRV features from a modulated signal among the candidate modulated signals.

Specifically, the system 10 for detecting stress may use a feature extractor to extract multiple second HRV features (also referred to as a set of second HRV features) from a candidate modulated signal.

In some implementations, when the feature extractor is used to extract multiple HRV features (e.g., second HRV features) from a signal (e.g., a candidate modulated signal), the signal may be segmented into multiple segments of a predefined length, and multiple HRV features (e.g., 61) may be extracted from each segment. Then, the feature extractor may perform statistical aggregation on the HRV features to derive 13 statistical values for each feature. Thus, one signal may correspond to 793 statistical features. Finally, the feature extractor may use a method, such as Recursive Feature Elimination (RFE) to select multiple features from the 793 statistical features as the final output HRV features.

For example, the feature extractor may segment a 24-hour candidate modulated signal into 288 segments, each 5 minutes long, and extract 61 HRV features from each segment. The extracted features may include 8 time-domain features, 6 frequency-domain features, and 47 multi-scale features. After aggregating the 61 HRV features from each of the 288 segments, 13 statistical values per HRV feature may be obtained, yielding 793 (e.g., 61*13) statistical features per candidate modulated signal. The feature extractor may then use a random forest algorithm to select the most important 100 features as the multiple (e.g., one set of) second HRV features for the candidate modulated signal.

Examples of the 8 time-domain features may include: a mean NN interval (meanNN), standard deviation of NN intervals (sdNN), a coefficient of variation, a first-order difference mean, root mean square of successive differences (RMSSD), a standard deviation of absolute first-order differences, proportion of NN intervals greater than 50 ms (pNN50), and a normalized mean of absolute first-order differences.

Examples of the 6 frequency-domain features may include: a high-frequency (HF) power, a low-frequency (LF) power, a HF/LF power ratio, a normalized HF power, and a very low frequency (VLF) power.

Examples of the 47 multi-scale features may include: multi-scale permutation entropy (MSPE), multi-scale modified permutation entropy (MSmPE), permutation entropy (PE), a first-order difference, a second-order difference, differences of first- and second-order differences, a sum of first- and second-order differences, and a total asymmetry index, which may be calculated based on RR intervals and the differences of the RR intervals (dRR).

In some implementations, the system 10 for detecting stress may extract multiple sets of second candidate HRV features by using a feature extractor for each candidate modulated signal. Then, the system 10 may select one set from among multiple sets of second candidate HRV features using a selection model. For example, as shown in FIG. 5, the system 10 for detecting stress may use a selection model 54 to choose from among multiple sets of second candidate HRV features corresponding to multiple candidate modulated signals 53 in order to obtain the multiple (e.g., one set of) second HRV features 55 corresponding to a modulated signal. In other implementations, the system 10 may use the selection model to choose one of the candidate modulated signals as the modulated signal, and then extract the corresponding (e.g., set of) second HRV features from the modulated signal using the feature extractor.

In some implementations, the selection model 54 may be a convolutional neural network (CNN)-based model that outputs a corresponding weight for each set of second HRV features with input of multiple sets of second HRV features. The system 10 for detecting stress may select the set of second HRV features with the highest weight as the second HRV features 55.

Advantageously, once trained, the selection model 54 may identify the most appropriate modulation scheme for cardiovascular physiological signals from different individuals or scenarios. In contrast to the first HRV features (e.g., identity features), the selected second HRV features are less susceptible to individual or scenario differences and may be considered task-related features. Therefore, in the present disclosure, the (e.g., set of) second HRV features 55 selected are also referred to as content features.

In operation S430, the system 10 for detecting stress may determine the stress detection result 13 corresponding to the cardiovascular physiological signal 12 based on the multiple first HRV features and the multiple (e.g., one set of) second HRV features.

Specifically, obtaining first HRV features that reflect identity provides additional individual-related information, and obtaining second HRV features that are insensitive to individual or scenario differences provides task-related information. With the aid of both types of additional information, the system may perform a more accurate analysis of the target task. In other words, the system 10 for detecting stress may obtain a more accurate stress detection result 13.

For example, as shown in FIG. 5, the system 10 for detecting stress may combine the multiple first HRV features 52 and the multiple second HRV features 55, as input to a multi-head attention model 56, to generate embeddings 57 (or an embedding space). The embeddings 57 may then be used by a classifier 58 to produce the stress detection result 13.

For example, the multi-head attention model 56 may be a neural network that includes an input layer, an attention layer, a fusion layer, a linear layer, and the like. The classifier 58 may be, for example, a temporal convolutional neural network (TCNN) classifier.

As shown in FIG. 5, the architecture for implementing the stress detection method may include at least the selection model 54, the multi-head attention model 56, and the classifier 58, all of which may be trained simultaneously. Using an existing dataset (e.g., TILES 2018), the system 10 for detecting stress may train these machine learning models using a guided contrastive learning approach.

Specifically, to enable the multi-head attention model 56 to push features of similar identities apart and pull features of dissimilar identities close, an identity contrastive loss LID may be defined as:

$${L}_{ID} = \frac{1}{α} log ( 1 + {\sum }_{j} exp ( - α ( {S}_{\text{?}} - m ) ) ) + \frac{1}{β} log ( 1 + {\sum }_{k} exp ( β ( {S}_{\text{?}} - m ) ) ) ,$$

$$\text{?} \text{indicates text missing or illegible when filed}$$

where S may represent the similarity (e.g., cosine similarity) between different samples, i may denote the sample index, k and j may represent the indices of the positive and negative sample pairs, respectively, and m, α, and β may be hyperparameters. Specifically, the hyperparameter m may be used to push apart the distributions of positive and negative samples, while the hyperparameters α and β may be used to adjust the sensitivity of the loss to positive and negative samples.

Next, to take into account the classification performance of stress detection, a stress contrastive loss L_{Stress} may be defined by:

$${L}_{Stress} = - \frac{1}{N} {\sum }_{i = 1}^{N} log ( \frac{{\sum }_{j = 1}^{N} {\text{?}}_{\{ \text{?} \}} exp ( \frac{\text{?}}{τ} )}{{\sum }_{j = 1 , j \neq i}^{N} exp ( \frac{\text{?}}{τ} )} ) ,$$

$$\text{?} \text{indicates text missing or illegible when filed}$$

where N may denote the total number of samples, sᵢ and sⱼ may be the stress labels of samples i and j, respectively, [symbol] may be an indicator function (e.g., equal to 1 when sᵢ=sⱼ, and 0 otherwise), zᵢ and zⱼ may be the feature vectors of samples i and j, respectively, and t may be a temperature coefficient.

Then, during the fine-tuning stage, a binary cross entropy loss L_{BCE} may be used and defined by:

$${ℒ}_{BCE} = - \frac{1}{N} {\sum }_{i = 1}^{N} ⌈ {y}_{i} log ( {\hat{y}}_{\text{?}} ) + ( 1 - {y}_{i} ) log ( 1 - {\hat{y}}_{\text{?}} ) ⌋ ,$$

$$\text{?} \text{indicates text missing or illegible when filed}$$

where N may denote the total number of samples, yᵢ may be the ground truth label of sample i, and ŷᵢ may be the predicted probability of stress of sample i (e.g., between 0 and 1).

To emphasize the purpose of each training stage in the guided contrastive learning process, weights may be introduced into the total loss Lₜₒₜₐₗ, which is defined by:

$${ℒ}_{total} = α \cdot {ℒ}_{ID} + β \cdot {ℒ}_{Stress} + γ \cdot {ℒ}_{BCE}$$

During actual training, in the early training phase (e.g., the first n epochs), the weights (α, β, γ) may be set to (0.6, 0.3, 0.1). Advantageously, using a higher α value may ensure disentanglement of individual or identity features, and adjusting the β value may control the retention of task-related features. During the fine-tuning stage in the later phase of training (e.g., starting from epoch n+1), the weights (α, β, γ) may be set to (0.1, 0.0, 0.9). Advantageously, allowing the γ value to dominate during the fine-tuning stage may ensure the accuracy of the classifier 58. In some implementations, the selection model 54 and the multi-head attention model 56 may be frozen in the later phase of training, focusing on fine-tuning the classifier 58.

After training the various models, the system 10 for detecting stress may perform highly accurate stress detection using the architecture illustrated in the example of FIG. 5.

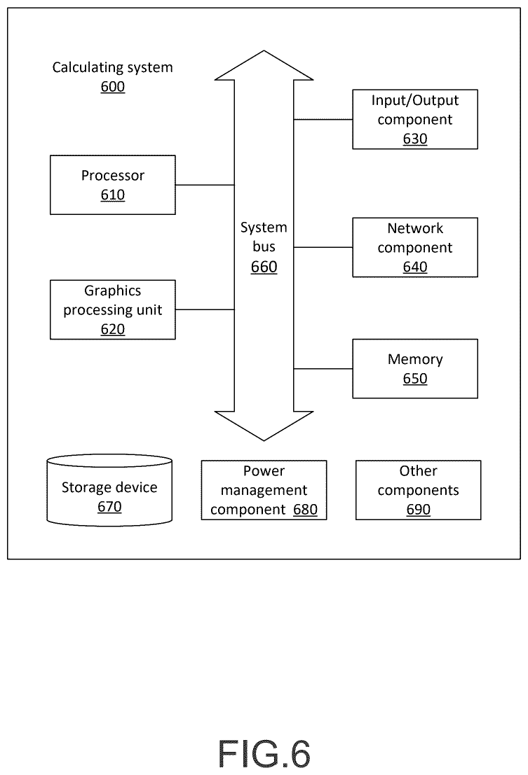

FIG. 6 is a block diagram illustrating a calculating system, according to an example implementation of the present disclosure.

Referring to FIG. 6, the method for detecting stress introduced herein may be implemented on one or more calculating systems 600 having various hardware components. For example, the calculating system 600 may implement the first device 11, the system 10 for detecting stress, and/or the second device 14. In some implementations, the calculating system 600 may, for example, be implemented in the form of an electronic device, which for example includes, but is not limited to, one or more of the following components: a processor (Central Processing Unit, CPU) 610, a graphics processing unit (GPU) 620, input/output components 630, network components 640, and memory 650. These components may communicate and transmit data via a system bus 660. However, the present disclosure does not limit the specific model, quantity, and configuration of individual components herein. A person of ordinary skill in the art may adjust, select, or add/remove components according to the specific needs and operating environment when implementing the present disclosure.

In some implementations, the main computing core within the calculating system 600 may be one or more processors 610. This processor 610 may be responsible for the main computing processes and related control logic for algorithms, such as deep learning. In some implementations, the processor 610 may be configured to execute processing instructions (e.g., machine-executable instructions) stored in a non-volatile/transitory computer-readable medium (e.g., a storage device 670).

In some implementations, to improve the computing efficiency of deep learning, the calculating system 600 may also include one or more graphics processing units 620 specifically designed for performing large-scale parallel computing. This graphics processing unit 620 may effectively enhance the computing capability of the system when performing deep learning training and inference.

In some implementations, the calculating system 600 may include a variety of input/output components 630 for receiving user input and displaying system output. For example, the input/output components 630 may include a keyboard, a mouse, a touchpad, a display screen, speakers, and other types of sensing devices.

In some implementations, the calculating system 600 may also include network components 640 for network communication. For example, these network components 640 may include network interface cards for wired or wireless network connections, or communication modules for 3G, 4G, 5G, or other wireless communication technologies.

In some implementations, the calculating system 600 may include one or more memories 650, such as volatile memory components like random access memory (RAM). The memory 650 may be used to store deep learning model parameters, as well as other data and programs used to run deep learning algorithms and the like. In some implementations, the memory 650 may, for example, store a feature extractor.

Furthermore, the calculating system 600 may also include one or more of the following components: a storage device 670, power management components 680, and various other components 690.

In some implementations, the calculating system 600 may include one or more storage devices 670, such as non-volatile memory components like a hard disk drive (HDD) or a solid state drive (SSD). These storage devices 670 may be used to store deep learning software code, training data, model parameters, and other information. Additionally, the storage devices 670 may also be used to store intermediate results and final output of deep learning algorithms and the like.

In some implementations, the calculating system 600 may include one or more power management components 680 for providing power to the various hardware components of the calculating system 600 and managing their power consumption. These power management components 680 may include batteries, power converters, and other power management devices.

In some implementations, the calculating system 600 may also include various other (hardware) components 690, such as cooling fans, heat sinks, and various other control and monitoring devices. The present disclosure does not limit to these examples herein.

Furthermore, the implementations herein may also be implemented as one or more computer program products, which include a computer program having one or more instructions. Specifically, a computer program (also referred to as a program, software, script, or code) may be presented in any form of programming language, and this computer program may be deployed in any form. During the operation of the calculating system 600 (e.g., an electronic device), the instructions or a part thereof may also reside entirely or at least partially within the processor 610, thus allowing the processor 610 to perform the methods introduced herein accordingly.

In summary, the method and system for detecting stress provided in the implementations of the present disclosure may effectively decouple cardiovascular physiological signals, thereby improving the accuracy of stress detection.

In view of the present disclosure, various techniques may be used for implementing the disclosed concepts without departing from the scope of those concepts. Moreover, while the concepts have been disclosed with specific reference to certain implementations, a person of ordinary skill in the art may recognize that changes may be made in form and detail without departing from the scope of those concepts. As such, the disclosed implementations are considered in all respects as illustrative and not restrictive. It should also be understood that the present disclosure is not limited to the specific implementations disclosed. Still, many rearrangements, modifications, and substitutions are possible without departing from the scope of the present disclosure.

What is claimed is:

1. A method for detecting stress based on cardiovascular physiological signals, the method comprising:
obtaining a plurality of signal segments based on a cardiovascular physiological signal, the plurality of signal segments being stationary, as determined by an augmented Dickey-Fuller (ADF) test;
calculating a plurality of trends for the plurality of signal segments;
obtaining a detrended signal corresponding to the cardiovascular physiological signal based on the plurality of trends;
extracting a plurality of first heart rate variability (HRV) features based on the detrended signal; and
obtaining a stress detection result corresponding to the cardiovascular physiological signal based on the plurality of first HRV features.

2. The method of claim 1, further comprising:
obtaining a plurality of candidate modulated signals, based on the cardiovascular physiological signal, using a plurality of modulation schemes;
obtaining a plurality of second HRV features extracted from a modulated signal selected from the plurality of candidate modulated signals; and
obtaining the stress detection result corresponding to the cardiovascular physiological signal based on the plurality of first HRV features and the plurality of second HRV features.

3. The method of claim 2, wherein obtaining the plurality of second HRV features extracted from the modulated signal selected from the plurality of candidate modulated signals comprises:
extracting a plurality of second candidate HRV features respectively from the plurality of candidate modulated signals;
obtaining a plurality of weights corresponding to the plurality of second candidate HRV features using a selection model; and
selecting one of the plurality of second candidate HRV features that corresponds to a maximum weight of the plurality of weights,
wherein the selected one of the plurality of second candidate HRV features is extracted from the modulated signal.

4. The method of claim 2, wherein each of the plurality of modulation schemes comprises at least one of magnitude warping, scaling, noise addition, jittering, time warping, window warping, and permutation.

5. The method of claim 2, wherein obtaining the stress detection result corresponding to the cardiovascular physiological signal based on the plurality of first HRV features and the plurality of second HRV features comprises:
obtaining embeddings using a multi-head attention model based on the plurality of first HRV features and the plurality of second HRV features; and
obtaining the stress detection result based on the embeddings.

6. The method of claim 5, wherein the multi-head attention model is trained using a guided contrastive learning method.

7. The method of claim 1, wherein obtaining the plurality of signal segments based on the cardiovascular physiological signal comprises:
normalizing the cardiovascular physiological signal to obtain a normalized signal;
segmenting the normalized signal into a plurality of first candidate segments based on a first segment length;
determining whether all of a plurality of first p-values, each corresponding to one of the plurality of first candidate segments, are smaller than a predefined threshold using the ADF test;
determining that the plurality of first candidate segments is the plurality of signal segments when all of the plurality of first p-values are smaller than the predefined threshold; and
re-segmenting the normalized signal into a plurality of second candidate segments using a second segment length when at least one of the plurality of first p-values is not smaller than the predefined threshold.

8. The method of claim 7, wherein the first segment length is shorter than the second segment length.

9. The method of claim 1, wherein calculating the plurality of trends for the plurality of signal segments comprises:
fitting the plurality of trends, based on the plurality of signal segments, using an ordinary least squares (OLS) method.

10. A system for detecting stress based on cardiovascular physiological signals, the system comprising:
at least one processor; and
at least one non-transitory computer-readable medium coupled to the at least one processor and storing one or more computer-executable instructions that, when executed by the at least one processor, cause the system to:
obtain a plurality of signal segments based on a cardiovascular physiological signal, the plurality of signal segments being stationary, as determined by an augmented Dickey-Fuller (ADF) test;
calculate a plurality of trends for the plurality of signal segments;
obtain a detrended signal corresponding to the cardiovascular physiological signal based on the plurality of trends;
extract a plurality of first heart rate variability (HRV) features based on the detrended signal; and
obtain a stress detection result corresponding to the cardiovascular physiological signal based on the plurality of first HRV features.

11. The system of claim 10, wherein the one or more computer-executable instructions that, when executed by the at least one processor, further cause the system to:
obtain a plurality of candidate modulated signals, based on the cardiovascular physiological signal, using a plurality of modulation schemes;
obtain a plurality of second HRV features extracted from a modulated signal selected from the plurality of candidate modulated signals; and
obtain the stress detection result corresponding to the cardiovascular physiological signal based on the plurality of first HRV features and the plurality of second HRV features.

12. The system of claim 11, wherein obtaining the plurality of second HRV features extracted from the modulated signal selected from the plurality of candidate modulated signals comprises:
extracting a plurality of second candidate HRV features respectively from the plurality of candidate modulated signals;
obtaining a plurality of weights corresponding to the plurality of second candidate HRV features using a selection model; and
selecting one of the plurality of second candidate HRV features that corresponds to a maximum weight of the plurality of weights,
wherein the selected one of the plurality of second candidate HRV features is extracted from the modulated signal.

13. The system of claim 11, wherein each of the plurality of modulation schemes comprises at least one of magnitude warping, scaling, noise addition, jittering, time warping, window warping, and permutation.

14. The system of claim 11, wherein obtaining the stress detection result corresponding to the cardiovascular physiological signal based on the plurality of first HRV features and the plurality of second HRV features comprises:
obtaining embeddings using a multi-head attention model based on the plurality of first HRV features and the plurality of second HRV features; and
obtaining the stress detection result based on the embeddings.

15. The system of claim 14, wherein the multi-head attention model is trained using a guided contrastive learning method.

16. The system of claim 10, wherein obtaining the plurality of signal segments based on the cardiovascular physiological signal comprises:
normalizing the cardiovascular physiological signal to obtain a normalized signal;
segmenting the normalized signal into a plurality of first candidate segments based on a first segment length;
determining whether all of a plurality of first p-values, each corresponding to one of the plurality of first candidate segments, are smaller than a predefined threshold using the ADF test;
determining that the plurality of first candidate segments is the plurality of signal segments when all of the plurality of first p-values are smaller than the predefined threshold; and
re-segmenting the normalized signal into a plurality of second candidate segments using a second segment length when at least one of the plurality of first p-values is not smaller than the predefined threshold.

17. The system of claim 16, wherein the first segment length is shorter than the second segment length.

18. The method of claim 10, wherein calculating the plurality of trends for the plurality of signal segments comprises:
fitting the plurality of trends, based on the plurality of signal segments, using an ordinary least squares (OLS) method.
